(19) United States
(12) Patent Application Publication
(10) Pub. No.: US 2010/0137864 A1
(43) Pub. Date: Jun. 3, 2010

(54) Percutaneous radial pin

(76) Inventor: Dominique Persoons, Sarrebourg (FR)

Correspondence Address: THE WATSON INTELLECTUAL PROPERTY GROUP, PLC, 3133 HIGHLAND DRIVE, SUITE 200, HUDSONVILLE, MI, 49426, US

(21) Appl. No.: 12/380,290

(22) Filed: Feb. 25, 2009

(30) Foreign Application Priority Data
Feb. 25, 2008 (FR) 0851182

(51) Int. Cl. A61B 17/56

(52) U.S. Cl. 606/64

(57) ABSTRACT

The present invention has as its object a percutaneous radial pin. A pin, characterized in that it comprises a proximal locking device (2) and a distal locking device (3), is of a non-fixed diameter, and has a different elasticity at its two ends. The invention is most particularly applicable to the area of bone surgery, particularly for the treatment of fractures of long bones and, in particular, of radius fractures.

The present invention relates to the area of bone surgery, particularly the treatment of fractures of long bones and, in particular, to fractures of the radius, and has as its object a pin for fractures of the radius with proximal and distal locking.

At the present time, fractures of the distal radius are very often associated with a comminution of the bone, particularly in older persons. Treatments by means of plaster cast or an aligning connection often result in a secondary compression fracture of the fracture and a shortening of the radius. This results in a conflict at the level of the distal radio-ulnar joint, which causes pain and stiffness.

Through the use of the modern techniques of osteosynthesis, it has been attempted to maintain the length of the radius despite the comminution of the bone. For this purpose, two techniques may be used, namely, anterior or posterior plate osteosynthesis and osteosynthesis by means of an external fixing device bridging the wrist joint (ligamentotaxis).

Osteosynthesis has the disadvantage of an open center when there are numerous bone fragments, and the quality of the bone is not always favorable for a solid fixation of the screws. The external fixing device (ligamentotaxis), on the other hand, entails a stiffening of the wrist.

The current percutaneous pinning device has the advantage of making it possible to preserve a technique with a closed center, which facilitates its use. This technique is therefore comparable to external fixation (ligamentotaxis). However, the pin that is used brings about a local osteosynthesis which does not require the bridging of the wrist joint, in such a way that a stiffening of the latter may be avoided. Such a device therefore likewise makes it possible to preserve the qualities of osteosynthesis with an open center.

The present invention has the objective of eliminating the disadvantages of the modern techniques of osteosynthesis by means of a plate or fixing device, and of improving the current device by means of pinning, by proposing a pin that makes it possible to maintain the length of the radius.

For this purpose, the pin in accordance with the invention is characterized in that it comprises a proximal locking device and a distal locking device, and has a different elasticity at its two ends.

The invention will be better understood by means of the description in the following, which relates to one preferred mode of implementation, which is provided by way of example and is not limitative, and is explained by reference to the appended schematic diagrams, in which:

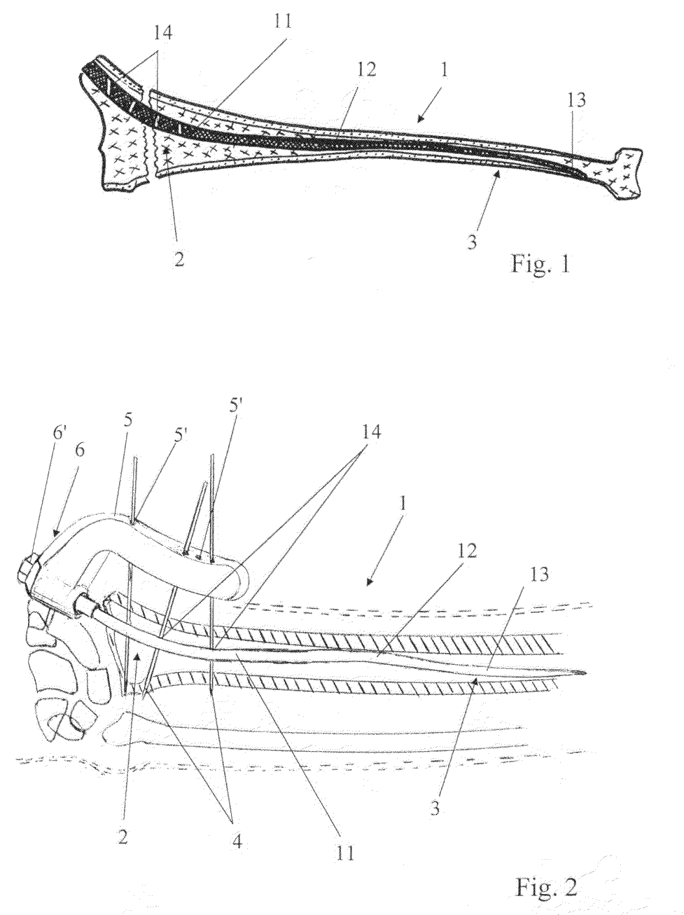

FIG. 1: Is a partial cross-section depicting a pin in accordance with the invention inserted into its service position, without its ancillary unit, and:

FIG. 2: Is a partial view analogous to that of FIG. 1 and depicts the pin with its ancillary unit locked in position by means of pins.

FIGS. 1 and 2 of the appended diagrams depict a radius fractured at its distal end and provided with a percutaneous radial pin 1.

In accordance with the invention, this percutaneous radial pin 1 comprises a proximal locking device 2 and a distal locking device 3, is of non-fixed diameter, and has a different elasticity at its two ends.

In accordance with one characteristic of the invention, the percutaneous radial pin 1 is made of an elastic metallic alloy, has an essentially elongated S-shape, and comprises three parts, namely: a first rigid curved part 11 on its proximal quarter which forms the proximal locking device 2; a second straight part 12; and a supple curved and tapered distal end 13 which forms the distal locking device 3.

The first curved part 11 on the proximal quarter extends over a length of approximately 5 cm at an inclination of +15°±1.5°. Such an inclination corresponds to the angulation of the radial styloid in relation to the axis of the bone, in such a way that it facilitates the introduction of the percutaneous radial pin 1. The supple curved and tapered distal end 13 has an inclination of +10°±5°, and reproduces the curvature of the proximal diaphysis of the radius. This supple curved and tapered distal end 13 is intended, in addition, to allow a significant elasticity of the distal quarter of the percutaneous radial pin 1, in order to facilitate the introduction of the said pin 1 into any anatomy whatsoever and, finally, to be fixed into the narrow proximal part of the medullar channel.

The diameter of the percutaneous radial pin 1 decreases along its entire length in a conical manner—that is to say, from the proximal part up to the distal part—, and varies between 5 mm±1 mm and 1.5 mm±1 mm.

The length of the percutaneous radial pin 1 is advantageously 14 cm±3 cm, as a function of the sizes. This length is determined in such a manner that the percutaneous radial pin 1 is implanted in hard contact and provides a tight contact with the cortex of the diaphysis of the bone, with this being intended to bring about a fixation at three points. This length of the percutaneous radial pin 1, in combination with the elasticity of the pin itself and its elongated S-shape, makes it possible for the percutaneous pin 1 to be self-stabilizing at the level of the diaphysis of the bone.

The auto-stability of the percutaneous radial pin 1 is essential, and is obtained by means of the specific form of the pin in an elongated S shape which is made from an elastic metallic alloy, the distal end of which is tapered.

In accordance with another characteristic of the invention, the percutaneous radial pin 1 advantageously has, on the first curved part 11 of its proximal quarter close to its proximal end, a certain number of non-parallel transversal holes 14 for the fixation of transversal locking pins 4 along the entire length of the radius. These pins 4, which advantageously number between 3 and 7, are intended to block the distal end of the percutaneous pin 1 and to prevent the telescoping of the fracture. The pins 4 preferably have a diameter of 2 mm±0.4 mm, and are simply driven into the bone of the fractured zone after passage through the non-parallel transversal holes 14. Because the pins are driven into the bone in a non-parallel manner, their stability in the locking position is reinforced.

In accordance with one characteristic of the invention, the percutaneous radial pin 1 can be advantageously provided with a complementary locking hole (not depicted) for fixing and guiding a pin bridging the radio-carpal joint through cooperation with the bones of the carpus.

The invention likewise has as its object an ancillary unit for the implementation of the percutaneous radial pin 1, which is composed of an external locking frame 5 in the form of an “L”, the shortest branch of which is provided with fixation means 6 for clamping the proximal end of the percutaneous radial pin 1, and the longest branch of which has a curve corresponding to the first curved part 11 of the proximal quarter of the said percutaneous radial pin 1, extends in parallel to this curved part 11, and is provided with passage and guiding holes 5′ for the transversal locking pins 4.

The passage and guiding holes 5′ for the transversal locking pins 4 are, in the service position of the external locking frame 5, aligned with the non-parallel transversal holes 14 of the first curved part 11 of the proximal quarter of the percutaneous radial pin 1 on the proximal end of the said percutaneous radial pin 1.

The fixation means 6 for clamping the external locking frame 5 on the proximal end of the percutaneous radial pin 1 consists of a clamping device that can be moved by means of a button 6′. Such a device is not represented in detail and is of known composition—that is to say, it has a hollow part in which the proximal end of the percutaneous radial pin 1 cooperating with a forged part solidly connected with the operating button 6′ fits closely, with the hollow part advantageously forming a maintenance and guiding support cooperating with the end part of the corresponding percutaneous radial pin 1, in which the forged part of the operating button 6′ is tightened. Thus, during the tightening of the operating button 6′, the proximal end of the percutaneous radial pin 1 is drawn by the means 6 in such a manner that the pin 1 locks perfectly with the locking frame 5.

Thus, after the insertion of the percutaneous radial pin 1 into the medullar channel of the radius and its precise positioning in this channel, the pins 4 are inserted by guiding through the passage and guiding holes 5′ of the external locking frame 5, through the non-parallel transversal holes 14 of the first curved part 11 of the proximal quarter of the percutaneous radial pin 1, in order to be driven into the bone of the fractured zone to ensure the stabilization of this said fractured zone.

The pins 4 are put in place percutaneously, and are then cut level with the skin. These pins 4 prevent the telescoping of the fracture and, because of their placement in a non-parallel manner, their stability in the service position is increased. After the consolidation of the fracture, the percutaneous radial pin 1 is immovable after the removal of the pins percutaneously 4.

By means of the invention, it is possible to provide a percutaneous radial pin 1 for radius fractures which is easily implantable and requires a simple ancillary unit. This pin 1 perfectly embodies the form of a radial styloid, and its locking can be carried out by means of simple pins 4, without the use of special screws.

Moreover, the shape of the pin 1 with a double curve allows a fixation at three points of hard contact in the medullar cavity of the radius.

The pin in accordance with the invention can be implanted in the distal radius with a closed center through the aperture of the radial styloid, and is self-stabilizing after percutaneous locking. This pin stabilizes the external column of the distal radius, while the column is, in accordance with Reggazonni and Rikki, fixed internally by the locking pins 4.

The invention is not, of course, limited to the mode of implementation described and represented in the attached diagrams. Modifications are still possible, particularly in regard to the composition of the various elements or the substitution of equivalent techniques, without thereby leaving the range of protection of the invention.

1. A percutaneous radial pin (1), characterized in that, it is made of an elastic metallic alloy, in that, it has an essentially elongated S-shape and comprises three parts, namely, a first curved part (11) on its proximal quarter, which forms a proximal locking device (2), a second straight part (12), and a supple curved and tapered distal end (13) forming a distal locking device (3), and in that, it has a diameter that decreases along its entire length, that is to say, from the proximal part to the distal part, and has a different elasticity at its two ends, with the double curve, which is due to the S-form, bringing about a fixation at three points of hard contact in the medullar cavity of the radius.

2. A pin in accordance with claim 1, characterized in that, the first curved part (11) on the proximal quarter extends for a length of approximately 5 cm at an inclination of +15°±5°.

3. A pin in accordance with claim 1, characterized in that, the supple curved and tapered distal end (13) has an inclination of +10°±5°, and reproduces the curvature of the proximal diaphysis of the radius.

4. A pin in accordance with claim 1, characterized in that, the tapered distal end (13) has a significant elasticity and is fixed in the narrow proximal part of the medullar channel.

5. A pin in accordance with claim 1, characterized in that, its diameter decreases along its entire length in a conical manner, that is to say, from the proximal part to the distal part, and ranges between 5 mm±1 mm and 1.5 mm±1 mm.

6. A pin in accordance with claim 1, characterized in that, its length is 14 cm±3 cm, as a function of the sizes.

7. A pin in accordance with claim 1, characterized in that, it has a certain number of non-parallel transversal holes (14) close to its proximal end on the first curved part (11) of its proximal quarter for the fixation of the transversal locking pins (4) along the entire length of the radius.

8. A pin in accordance with claim 7, characterized in that, it is provided with a complementary locking hole for fixing and guiding a pin bridging the radio-carpal joint through cooperation with the bones of the carpus.
